(19) United States
(12) Patent Application Publication
(10) Pub. No.: US 2007/0016136 A1
(43) Pub. Date: Jan. 18, 2007

(54) Suction hand-piece device with variable control off/on valve

(76) Inventor: John C. Opie, Scottsdale, AZ (US)

Correspondence Address: Squire, Sanders & Dempsey L.L.P.;Two Renaissance Squire, Suite 2700, 40 North Central Avenue, Phoenix, AZ, 85004-4498, US

(21) Appl. No.: 11/436,308

(22) Filed: May 18, 2006

Related U.S. Application Data
(60) Provisional application No. 60/682,593, filed on May 18, 2005.

(51) Int. Cl. A61M 1/00

(52) U.S. Cl. 604/119; 604/540

(57) ABSTRACT

A new suction device is herein disclosed that includes a valve to regulate suction to the tip of a suction device. By an operator, such as a nurse or surgeon, manipulating one or two fingers, a valve can be turned or slid to either reduce the force of the suction or to stop all suction thus leading to a quieter and more peaceful operating room, or to open the suction in order to aspirate blood or fluid.

### RELATED APPLICATIONS

This application claims priority to U.S. Provisional Patent Application No. 60/682,593 filed on May 18, 2005 which is hereby incorporated by reference herein.

### FIELD OF THE INVENTION

The present invention relates to medical devices, and more particularly, to a surgical suction device.

### BACKGROUND OF THE INVENTION

Existing suction devices are widely available and widely used. Essentially every surgery utilizes some type of suction, for example in the throat and/or in the area(s) in the body in which the surgery is performed. Current suction devices generally work well when required to aspirate blood and fluid, however, they have a noticeable irritation in that the suction cannot be regulated or turned off while the sucker tip is or is not in use. When these suction devices have suction applied on the tip, the suction device whistles or hisses creating a noise that is undesirable in the operating room.

Patent literature describes a number of suction wands for use in surgical suction systems to remove debris from a surgical site. Certain suction hand-held devices are designed to return blood to a cardiotomy reservoir in open-heart surgery for example. These are high volume-low vacuum systems and do not generally make a hissing noise. Occasionally they make gurgling or bubbling noises. They are generally left on and create no noise problems. The vacuum for these filtered systems is a rotating pump head on the heart-lung machine. Such a suction probe has several inlet ports large enough to permit the entrance of debris particles, which pass into a cardiotomy reservoir filter system before the blood is returned to the patient. Such systems are filtered because they allow blood to be returned to the patient to reduce transfusion requirements. Still other systems are autotransfusers. In this situation a heparin drip is run down a separate line and mixed with the air being aspirated up the wand. In this situation closing the line simply allows the heparin to drip up the aspiration line and the noise of the suction can be obliterated by closing the valve and not impairing the suction device operation in any way. Other systems more widely used are high vacuum and high volume suction systems and do create white noise problems. A potential effect of leaving the quiet suction valve closed for lengthy periods might be blood clots occluding the suction tubing this can usually be cleared by irrigating the line with solution if it occludes. This is not generally a difficult problem to solve.

### SUMMARY OF THE INVENTION

A new suction device is herein disclosed that includes a valve to regulate suction to the tip of a suction device. By an operator, such as a nurse or surgeon, manipulating one or two fingers, a valve can be turned or slid to either reduce the force of the suction or to stop all suction thus leading to a quieter and more peaceful operating room, or to open the suction in order to aspirate blood or fluid.

Approximately 72-76 million operations are completed annually in the USA. And many more are completed around the world. Essentially every one of these surgeries requires suction. Typically the anesthesiologist has a disposable Yankauer suction to aspirate the mouth, and the surgeon has one or sometimes two disposable Yankauer suckers. With 2 or 3 suctions running close to one another, substantial white noise is generated, which can and does make for difficult conversation. Some require non-general purpose and specialized suction and do not need a “quiet suction” system. All of the others would benefit for a hand controlled valve that can either reduce the force of the suction vacuum at the distal end of the sucker so as to more precisely control the vacuum and lessen the risk of aspirating living tissue and while the sucker is not being used it is useful to have some system on the sucker handle to stop the persistent white noise generated by air whistling through the tip of the sucker.

### OBJECTS OF THE INVENTION

The principle object of the present invention is to provide, a sucker that can be occluded and un-occluded by a valve.

A second object of this invention is to provide a valved suction device that can infinitely down regulate the vacuum force so as to lessen the risk of aspirating living tissue up the sucker and thus lessen potential strangulation damage the living tissue and providing ease of removal by an ability to close off the suction while extracting the tissue.

A third object of this invention is to provide a valve that can be manipulated by the surgeon's fingers or thumb and will not get in the way or routine surgical procedures or vision.

A final object of this invention is to provide a keep-it-simple general-purpose suction that can be closed to eliminate unnecessary white noise in the otherwise quiet operation room.

### BRIEF DESCRIPTION OF THE DRAWINGS

FIG. 1 is a plan view of the “Quiet Suction” with valve in handle.

FIG. 2 is a rear view of the valve turned on. The wand lumen will be un-occluded with this setting.

FIG. 3 is a lateral view of the quiet suction valve turned on. The lumen will be open with this setting.

### DESCRIPTION OF THE EMBODIMENTS

Reference will now be made in detail to the present exemplary embodiments of the invention, examples of which are illustrated in the accompanying drawings.

This invention generally relates to a valved wand forming the hand-held section of a surgical suction system for the removal of debris from a surgical site. The reason for adding a regulator valve to the plastic wand is to reduce white noise due to an unregulated suction wand in the generally quiet operating room. A second advantage exists with a suction wand that can be disconnected from suction by closing the valve when tissue is inadvertently aspirated up into the wand when suction is high. Currently, is it necessary to either kink the tubing to interrupt the suction or disconnect the wand from the suction tubing to release the suction force and thus release the tissue to minimize damage to any aspirated tissue that is still attached to the body—a piece of omentum for example. A simple valve mechanism will enable the vacuum to be turned off quickly and when the suction wand is not in use the valve can be left off thus preventing the white nose generated by the hissing or whistling of the air through the open holes of the wand.

Any style or type of valve suitable for regulating suction may be used, and the valve could be at any location upstream (i.e., closer to the vacuum source) than the tip of the device used to aspirate blood or bodily fluids. Preferably the device used to aspirate blood or bodily fluids is a hand-held wand or probe.

Surgeons who spend many hours in the operating room know well the irritating white noise generated by an open sucker-aspirating wand. On many days the sucker noise seems louder than it does on other days and this effect might be related to the degree of difficulty of the surgical procedure. Many surgeons complete several maneuvers to reduce the noise: one common technique is to bury the aspiration wand under the drapes or in a side pocket. That generally reduces the noise about 50%. Another technique is to move the tip down near the operating room nurse or operating room technician. While that tends to reduce the noise about 50% with respect to the surgeon it does have some drawbacks. On very many occasions the noise now affects the operating room scrub tech or nurse who cannot clearly hear what the surgeon is asking for and instrument hand up mistakes occur leading to periodic irritation. Another technique widely used to eliminate the white noise effect is to kink the tubing and clamp it within the back of a clamp or some similar snap such as an Alice clamp. If the jaws of a snap are used from time to time the snap will break at the pivot point and an instrument is destroyed in an effort to control the white noise from the sucker. A clamped tube clearly eliminates the white noise effect completely but it also has drawbacks if the suction is needed instantly and some scrambling becomes necessary to locate the occluding clamp and release it before the suction returns. All of these minor but repetitive problems can be easily eliminated by having a “quiet sucker” with a simple two way finger tip controlled valve placed in the distal section of the sucker wand handle. Surgeons skilled in the art of surgery will immediately appreciate the valve of a “noiseless” suction that can be turned on and off on demand by the surgeon.

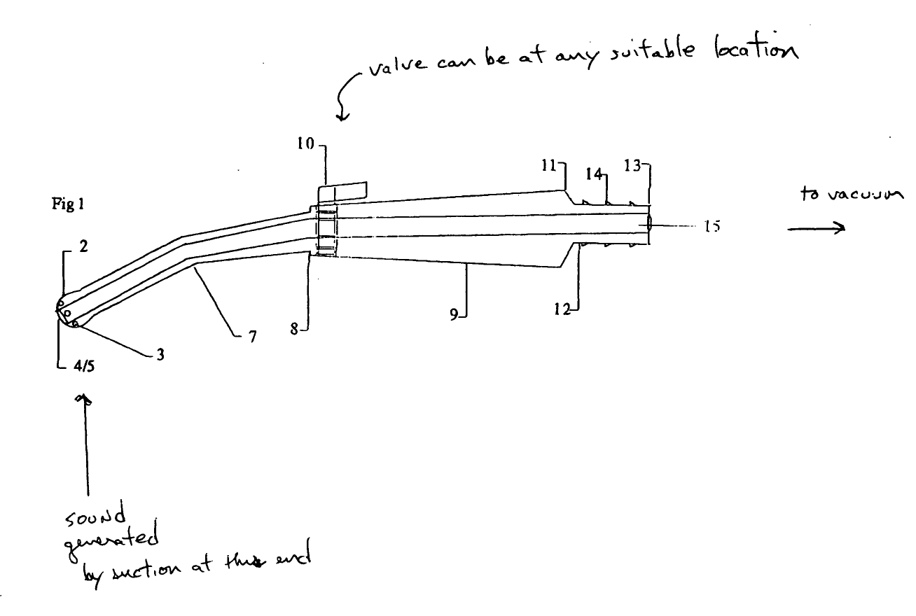

FIG. 1 shows the “Quiet Sucker.” The bulbous distal tip 2 has a multiplicity of square or round terminal side holes 3 arranged radially around the tip. The terminal end 4 of the tip has a single circular hole 5 sufficiently large enough to aspirate most surgical particles and fluids without being blocked too frequently.

The terminal tip may or may not be detachable by unscrewing or it may be part of the molded sucker wand it has a multiplicity of side holes 3 to prevent the terminal hole from becoming occluded. The body of the wand 7 may be angled, double angled or curved. The proximal end of the body of the wand 8 joins the distal end of the handle 9 and a two-way valve 10 is positioned here. The handle expands slightly as it ends proximally to comfortably fit the human hand. Several ridges on the handle may exist. (Not shown) These ridges are designed to reduce slipping and turning of the sucker while being held by offering a better grip since the sucker handle often becomes blood coated or wet or fat globule coated and thus may be slippery. The proximal aspect of the aspiration wand 11 ends in a circular contraction 12. The circular contraction extends proximally by about an inch before it ends 13. On its external surface are several small chevrons 14 designed to limit the likelihood of the suction tubing from slipping off the wand during use. A lumen 15 passes through the entire length of the wand and is the aspirating channel.

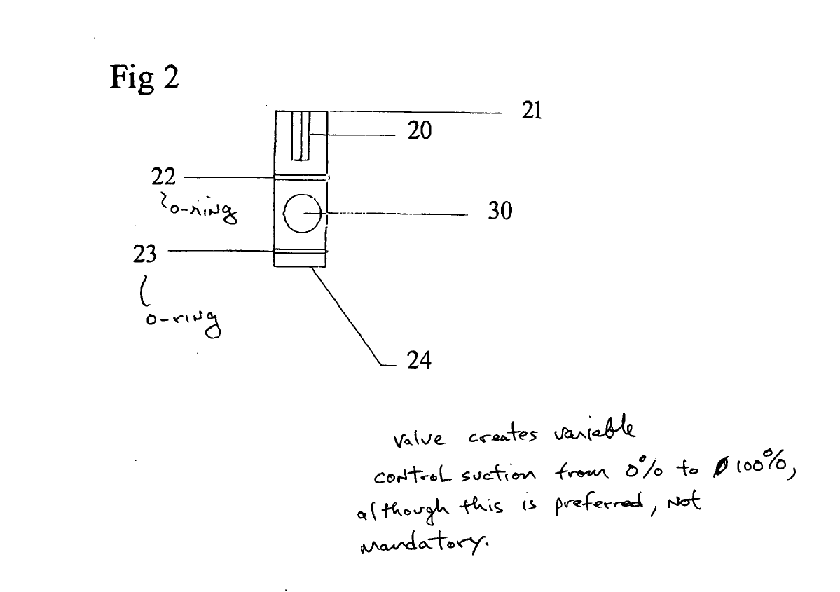

FIG. 2 is a rear view of the valve insert into the wand in an open position. The flag 20 of the valve 21 can be either in line with the suction wand thus the valve central hole 30 is open or at right angles to the wand thus being fully closed or at an infinite point between allowing variable suction. If the valve were turned into a right angle position the valve would be closed and all white noise will be stopped. There are two pressure O rings 22 and 23 to facilitate maintaining vacuum. The central round post of the valve is seen at 24. The round post passes through and is a snug fit with a matching vertical hole passing through the handle of the Quiet Suction wand.

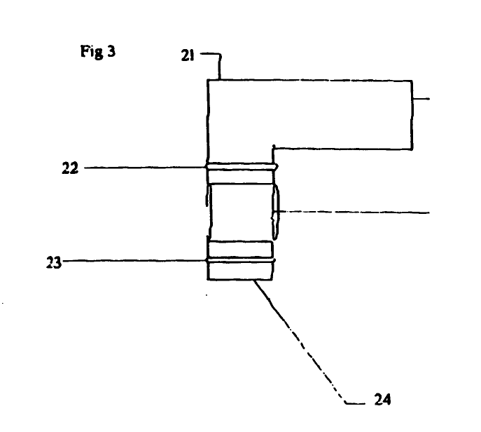

FIG. 3 is a lateral view of the valve. If the valve flag 20 is in this relationship with the wand the valve is open. A central hole 30 is now in inline connection with the lumen 15 of the wand (FIG. 1). The O rings 22 & 23 described previously in FIG. 2 can be seen. The central circular post 24 can be seen.

### OPERATION OF THE PREFERRED EMBODIMENT

Virtually all surgeries need operating room suction. Most of the time the aspirated material is discarded and does not need filtering. A simple, non-filtered, valved, hand held suction wand is described. Existing wands such as the Yankauer, while simple and effective, have one serious drawback in that while lying dormant the suction cannot be either down-regulated or turned off to reduce or eliminate white noise.

It is clear that several different valves could be used to regulate a Yankauer style suction wand. Some varieties have a hole where the valve is placed in the “Quiet Suction.” This proximal hole provides a way of reducing the chance of aspiration of tissue but it also allows continuous white noise to be generated through 2 holes in one suction wand.

A preferred embodiment of the invention is described with reference to FIGS. 1-3 of the drawings.

The design is simple and simple to operate. It is either on or off when a two-way stopcock is used.

### ALTERNATIVE EMBODIMENTS

It is understood the invention may have numerous alternative embodiments, varying certain features without departing from the scope of this invention, and some general descriptions of some alternate embodiments are set forth below.

A variation using a three-way stopcock could be used. In that variety if the flag is in line with the wand the lumen is closed but if the flag of the valve is to one or the other side the wand is open. By employing a three-way stopcock it is possible to have a lateral vent hole, however, if left open this side hole would once again generate white noise. The three-way stopcock is thus perhaps less desirable. It could be turned on to reduce the likelihood of clogging the connecting tubing. It is a little more complicated in that three positions of the valve would be possible. Open closed and vent hole open to reduce tubing clogging.

Two-and-fro valves or a sliding valve are potential alternative embodiments but they are somewhat more complicated to manufacture and assemble than the preferred embodiment. Different side hole tip configurations and sizes are possible. These include round holes, oval shaped or square holes.

While this invention has been described in terms of its preferred embodiments, those skilled in the art can appreciate that other modifications can be made without departing from the spirit and scope of this invention.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

What is claimed is:

1. A surgical wound suction wand (such as a Yankauer suction wand), wherein the wand has a tip, and that includes a valve to regulate the amount of suction at the tip.

2. A suction wand of claim 1 that contains a handpiece and a regulator valve in the hand-piece.

3. The wand of claim 1 that is configured to be handheld.

4. The wand of claim 1 that is plastic.

5. The wand of claim 1 that is not handheld.

6. The wand of claim 1 that has the structure of a Yankauer wand.

7. The wand of claim 2 that includes a valve therein.

8. A regulator valve situated upstream of the tip of the wand that can be switched off or on or to any position in between off and on so as to regulate the amount of vacuum applied to the tip of the wand.

9. The regulator valve of claim 8 that is positioned in the wand.

10. An aspirating wand that includes:
a) A bulbous tip containing multiple side round or square openings through which body debris can be aspirated.
b) A distal round opening.
c) A body long enough to comfortably fit inside any human body cavity.
d) A body that can be straight or angled
e) A body that has a distal end that connects to the bulbous tip
f) A body that has a proximal end that connects to the distal end of the handle of the wand.

11. A handle that includes.
a) A distal part that connects to the proximal end of the body of the wand.
b) A central section that is ergonomically shaped and will fit comfortably in the palm of the surgeon.
c) A distal section of the handle lying close to the position where the thumb and first finger lie that contains a valve to regulate or switch on or off the suction.
d) A proximal section of the wand with one or two chevrons that press fits on to regular suction tubing.

12. The regulator valve of claim 8 that opens or closes a throughway through the wand that is selected from the group consisting of:
a) A valve that is a simple push-push variety;
b) A valve that is one of several options of twist valves similar to the operation of a two-way Luer Lock™ valve with an external rotatable lever that opens or closes a lumen depending on whether it is (open) lined up with the long axis of the wand or (closed) at right angles to the long axis of the wand.

13. A surgical suction device comprising:
a suction wand having an internal passageway through which material may be aspirated when suction is applied to the internal passageway; and
a valve for regulating the amount of suction in the internal passageway.

14. The surgical suction device of claim 13 wherein the suction wand is substantially straight.

15. The surgical suction device of claim 13 wherein the suction wand has one or more curves and/or angles.

16. The surgical suction device of claim 13 wherein the suction wand includes a distal tip having one or more holes in fluid communication with the internal passageway.

17. The surgical suction device of claim 16 wherein the distal tip has a bulbous shape.

18. The surgical suction device of claim 16 wherein the one or more holes include a center hole positioned in the center of the distal tip.

19. The surgical suction device of claim 17 where in the center hole is round.

20. The surgical suction device of claim 16 wherein the one or more holes include one or more terminal side holes arranged radially around the distal tip.

21. The surgical suction device of claim 20 wherein the one or more terminal side holes are round.

22. The surgical suction device of claim 20 wherein the one or more terminal side holes are square.

23. The surgical suction device of claim 16 wherein the one ore more holes include a center hole positioned in the center of the distal tip and one or more terminal side holes arranged radially around the distal tip.

24. The surgical suction device of claim 16 wherein the distal tip is detachable from the suction wand.

25. The surgical suction device of claim 13 wherein the suction wand is a single unit.

26. The surgical suction device of claim 13 wherein the suction wand includes a handle.

27. The surgical suction device of claim 26 wherein the valve is positioned in the handle.

28. The surgical suction device of claim 26 wherein the suction wand has a distal end and a proximal end, the handle being positioned at the proximal end of the suction wand.
